(19) United States
(12) Patent Application Publication
(10) Pub. No.: US 2024/0306733 A1
(43) Pub. Date: Sep. 19, 2024

(54) INHALER ARTICLES HAVING FOLDED ENDS WITH UNIFORM APPEARANCE

(71) Applicant: PHILIP MORRIS PRODUCTS S.A., Neuchatel (CH)

(72) Inventor: Valerio D'Ambrogi, Roma (IT)

(21) Appl. No.: 18/681,672

(22) PCT Filed: Aug. 3, 2022
(86) PCT No.: PCT/EP2022/071848

§ 371 Date: Feb. 6, 2024

(30) Foreign Application Priority Data
Aug. 6, 2021 (EP) 21190038.6

(51) Int. Cl. A24F 42/80; A24F 42/20; A24F 42/60; A61M 15/00; B65G 47/84

(52) U.S. Cl. CPC A24F 42/80; A24F 42/20; A24F 42/60; A61M 15/0001; B65G 47/84

(57) ABSTRACT

The invention relates to a method for manufacturing inhaler articles, comprising steps of providing a semifinished inhaler article, providing a rotating drum comprising a plurality of slot elements with movable end faces, inserting the semifinished inhaler article into a slot element, providing an alignment element in proximity to the semifinished inhaler article, moving the movable end face of the slot element against the proximal end of the semifinished inhaler article to push the semifinished inhaler article along its longitudinal axis until the open distal end of the semifinished inhaler article contacts the alignment element, fixing the movable end face in position, providing a closing station in proximity to the semifinished inhaler article, and at least partially closing the distal end of the semifinished inhaler article. The invention further relates to packages of inhaler articles, to a rotating drum, and to an apparatus for manufacturing inhaler articles.

The present disclosure relates to a method for manufacturing inhaler articles. The present disclosure further relates to a package of inhaler articles with uniformly closed ends. The present disclosure further relates to a rotating drum for an apparatus for manufacturing inhaler articles. The present disclosure further relates to an apparatus for manufacturing inhaler articles.

In the field of manufacturing inhaler articles, it is known to provide a deformable tubular element and to fold a distal end of the deformable tubular element inwards by about 90 degrees to at least partially close the distal end of the deformable tubular element. Due to manufacturing tolerances in the production of the tubular elements, there may be small differences in length between individual tubular elements. These differences in lengths of the tubular elements may result in differing lengths of the distal portions of the semifinished inhaler articles that will be folded. This may lead to variations of the closed distal ends of the inhaler articles. This may lead to an uneven visual appearance of the closed distal ends of the inhaler articles. Similarly, variability in the relative longitudinal positions of the semifinished inhaler articles on a rotating drum may occur due to process turbulences, for example due to shifting of the semifinished articles onto the rotating drum. Such effects may similarly result in differing lengths of the distal portions of the semifinished inhaler articles that will be folded.

Variations of the closed distal ends of the inhaler articles may result in differences in the force which is required by a user to insert a finished inhaler article into a user-holder device. This could then lead to applying too much force and potentially disrupting the article. Generally, there may be limitations to the maximum bendable length of a paper tubular element due to its material properties. This may mean that the more the tube is bent, the more force has to be applied by a user when inserting it in to the user-holder device.

It would be desirable to provide a method and a device for reproducibly and automatically manufacturing an inhaler article. It would be desirable to provide a method and a device for manufacturing inhaler articles with a uniform appearance of the closed distal ends of the articles.

It would be desirable to provide a method and a device for manufacturing an inhaler article at sufficiently high speed.

It would be desirable to provide a method and a device for manufacturing an inhaler article, wherein the manufacturing method can be implemented in existing manufacturing lines used for production of inhaler articles.

It would be desirable to provide a method and a device for manufacturing an inhaler article that reduces risk of article rupture when being used with a user-holder device by a user.

According to an embodiment of the invention there is provided a method for manufacturing inhaler articles. The method may comprise providing a semifinished inhaler article comprising a longitudinal axis, a proximal end, and an open distal end. The method may comprise providing a rotating drum comprising a plurality of slot elements with movable end faces. The method may comprise inserting the semifinished inhaler article into a slot element. The method may comprise providing an alignment element in proximity to the semifinished inhaler article. The method may comprise moving the movable end face of the slot element against the proximal end of the semifinished inhaler article to push the semifinished inhaler article along its longitudinal axis until the open distal end of the semifinished inhaler article contacts the alignment element. The method may comprise fixing the movable end face in position. The method may comprise providing a closing station in proximity to the semifinished inhaler article. The method may comprise at least partially closing the distal end of the semifinished inhaler article.

According to an embodiment of the invention there is provided a method for manufacturing inhaler articles. The method comprises providing a semifinished inhaler article comprising a longitudinal axis, a proximal end, and an open distal end. The method comprises providing a rotating drum comprising a plurality of slot elements with movable end faces. The method comprises inserting the semifinished inhaler article into a slot element. The method comprises providing an alignment element in proximity to the semifinished inhaler article. The method comprises moving the movable end face of the slot element against the proximal end of the semifinished inhaler article to push the semifinished inhaler article along its longitudinal axis until the open distal end of the semifinished inhaler article contacts the alignment element. The method comprises fixing the movable end face in position. The method comprises providing a closing station in proximity to the semifinished inhaler article. The method comprises at least partially closing the distal end of the semifinished inhaler article.

By the step of moving the semifinished inhaler article such that its distal end contacts the alignment element, each processed article will be aligned such that the open distal ends of the semifinished inhaler articles are always located at the exact same height with respect to a direction parallel to their longitudinal axis before being at least partially closed. Thereby, effects of differences in lengths of individual articles due to manufacturing tolerances on the step of at least partially closing the distal ends of the articles may be compensated or reduced. Typical manufacturing tolerances of the lengths of the semifinished inhaler articles may be about +/−0.5 millimeter.

By the alignment of the heights of the semifinished inhaler articles, a method for reproducibly and automatically manufacturing inhaler articles is provided. By the alignment of the heights of the semifinished inhaler articles, a method for manufacturing inhaler articles with a uniform appearance of the at least partially closed distal ends of the articles is provided.

By providing articles with uniformly closed ends, a method and a device for manufacturing an inhaler article are provided that reduce risk of article rupture when being used with a user-holder device by a user.

A method for manufacturing an inhaler article at sufficiently high speed is provided. A method is provided which can be implemented in existing manufacturing lines used for the production of inhaler articles.

As used herein, the term ‘exact same height’ may refer to a difference in height of less than about 100 micrometers, preferably less than about 75 micrometers, and more preferably about 50 micrometers or less.

The movable end face of the slot element may be moved by means of a spring element coupled to the movable end face.

The method may comprise, after the step of at least partially closing the distal end of the semifinished inhaler article, steps of removing the semifinished inhaler article from the slot element and retracting the movable end face of the slot element. A retracting element may be provided for retracting the movable end face.

The step of removing the semifinished inhaler article from the slot element may comprise transferring the inhaler article to a second rotating drum which is reverse-orientated relative to the first rotating drum but which is otherwise identical to the first rotating drum.

The proximal end of the semifinished inhaler article may be an open proximal end, and the method may further comprise at least partially closing the proximal end of the semifinished inhaler article at the second rotating drum.

The semifinished inhaler article comprising an open distal end and an open proximal end may be a double-length inhaler article. The double-length inhaler article may comprise a double-length mouthpiece element and a double-length deformable tubular element. The double-length mouthpiece element is provided in the centre of the double-length deformable tubular element. After at least partially closing both ends, the double-length inhaler article may be cut in the middle to obtain two identical normal length inhaler articles. Manufacturing time can be significantly reduced by processing double-length inhaler articles.

The method may comprise, after the step fixing the movable end face in position, steps of, providing a filling station in proximity to the semifinished inhaler article and inserting a capsule into the open distal end of the semifinished inhaler article.

The filling station may be stationary and may be brought into proximity to the semifinished inhaler article by the rotation of the rotating drum. The filling station may be movable and may be brought into proximity to the semifinished inhaler article by moving the filling station towards the semifinished inhaler article.

The alignment element may be stationary and may be brought into proximity to the semifinished inhaler article by the rotation of the rotating drum. The alignment element may be movable and may be brought into proximity to the semifinished inhaler article by moving the alignment element towards the semifinished inhaler article.

The closing station may be stationary and may be brought into proximity to the semifinished inhaler article by the rotation of the rotating drum. The closing station may be movable and may be brought into proximity to the semifinished inhaler article by moving the closing station towards the semifinished inhaler article.

One or both of the filling station, the alignment element, and the closing station may be stationary or movable.

As used herein, the term ‘stationary’ may refer to a fixed position relative to the spatial position of the rotating drum.

As used herein, the terms ‘movable alignment element’, ‘movable filling station’, and ‘movable closing station’ may refer to a movable position relative to the spatial position of the rotating drum.

A movable element or station may be configured to advance, or rotate, towards the rotating drum in order to be brought into proximity with a semifinished inhaler article. A movable element or station may be configured to retract from, or rotate away from, the rotating drum after having processed the semifinished inhaler article.

A movable element or station may be configured to follow the rotation of the rotating drum. Thus, a movable element or station may be in a fixed position relative to a slot of the rotating drum when being in proximity to a semifinished inhaler article for processing the article.

The capsule may comprise nicotine.

The capsule may comprise a dry powder.

The inhaler article may comprise a capsule cavity for receiving the capsule. The capsule cavity may define a cylindrical space configured to contain a capsule. For example, the capsule may have an obround shape or a circular cross-section. The capsule cavity may have a substantially uniform or uniform diameter along the length of the capsule cavity. The capsule cavity may have a fixed cavity length. The capsule cavity has a cavity inner diameter, orthogonal to the longitudinal axis, and the capsule has a capsule outer diameter. The capsule cavity may be sized to contain an obround capsule. The capsule cavity may have a substantially cylindrical or cylindrical cross-section along the length of the capsule cavity. The capsule cavity may have a uniform inner diameter. The capsule may have an outer diameter that is about 80 percent to about 95 percent of the inner diameter of the capsule cavity. The configuration of the capsule cavity relative to the capsule may promote limited movement of the capsule during activation or piercing of the capsule.

The capsule cavity may be defined by the deformable element having a diameter in a range from about 6 millimetres to about 8 millimetres mm or about 6.6 millimetres.

The capsule may contain pharmaceutically active particles. For instance, the pharmaceutically active particles may comprise nicotine. The pharmaceutically active particles may have a mass median aerodynamic diameter of about 5 micrometres or less, or in a range from about 0.5 micrometres to about 4 micrometres, or in a range from about 1 micrometres to about 3 micrometres.

The capsule may contain nicotine particles comprising nicotine (also referred to as “nicotine powder” or “nicotine particles”) and optionally particles comprising flavour (also referred to as “flavour particles”). The capsule may contain a predetermined amount of nicotine particles and optional flavour particles. The capsule may contain enough nicotine particles to provide at least 2 inhalations or “puffs”, or at least about 5 inhalations or “puffs”, or at least about 10 inhalations or “puffs”. The capsule may contain enough nicotine particles to provide from about 5 to about 50 inhalations or “puffs”, or from about 10 to about 30 inhalations or “puffs”. Each inhalation or “puff” may deliver from about 0.1 mg to about 3 mg of nicotine particles to the lungs of the user or from about 0.2 milligrams to about 2 milligrams of nicotine particles to the lungs of the user or about 1 milligram of nicotine particles to the lungs of the user.

The nicotine particles may have any useful concentration of nicotine based on the particular formulation employed. The nicotine particles may have at least about 1 weight-percent nicotine up to about 30 weight-percent nicotine, or from about 2 weight-percent to about 25 weight-percent nicotine, or from about 3 weight-percent to about 20 weight-percent nicotine, or from about 4 weight-percent to about 15 weight-percent nicotine, or from about 5 weight-percent to about 13 weight-percent nicotine. Preferably, about 50 to about 150 micrograms of nicotine may be delivered to the lungs of the user with each inhalation or “puff”.

The capsule may hold or contain at least about 5 milligrams of nicotine particles or at least about 10 milligrams of nicotine particles. The capsule may hold or contain less than about 900 milligrams of nicotine particles, or less than about 300 milligrams of nicotine particles, or less than 150 milligrams of nicotine particles.

The capsule may hold or contain from about 5 milligrams to about 300 milligrams of nicotine particles or from about 10 milligrams to about 200 milligrams of nicotine particles.

When flavour particles are blended or combined with the nicotine particles within the capsule, the flavour particles may be present in an amount that provides the desired flavour to each inhalation or “puff” delivered to the user.

The nicotine particles may have any useful size distribution for inhalation delivery preferentially into the lungs of a user. The capsule may include particles other than the nicotine particles. The nicotine particles and the other particles may form a powder system.

The capsule may hold or contain at least about 5 milligrams of a dry powder (also referred to as a powder system) or at least about 10 milligrams of a dry powder. The capsule may hold or contain less than about 900 milligrams of a dry powder, or less than about 300 milligrams of a dry powder, or less than about 150 milligrams of a dry powder. The capsule may hold or contain from about 5 milligrams to about 300 milligrams of a dry powder, or from about 10 milligrams to about 200 milligrams of a dry powder, or from about 25 milligrams to about 100 milligrams of a dry powder.

The dry powder or powder system may have at least about 40 percent, or at least about 60 percent, or at least about 80 percent, by weight of the powder system comprised in nicotine particles having a particle size of about 5 micrometres or less, or in a range from about 1 micrometre to about 5 micrometres.

The particles comprising nicotine may have a mass median 5 aerodynamic diameter of about 5 micrometres or less, or in a range from about 0.5 micrometres to about 4 micrometres, or in a range from about 1 micrometre to about 3 micrometres or in a range from about 1.5 micrometres to about 2.5 micrometres. The mass median aerodynamic diameter is preferably measured with a cascade impactor.

The particles comprising flavour may have a mass median aerodynamic diameter of about 20 micrometres or greater, or about 50 micrometres or greater, or in a range from about 50 to about 200 micrometres, or from about 50 to about 150 micrometres. The mass median aerodynamic diameter is preferably measured with a cascade impactor.

The dry powder may have a mean diameter of about 60 micrometres or less, or in a range from about 1 micrometre to about 40 micrometres, or in a range from about 1.5 micrometres to about 25 micrometres. The mean diameter refers to the mean diameter per mass and is preferably measured by laser diffraction, laser diffusion or an electronic microscope.

Nicotine in the powder system or nicotine particles may be a pharmaceutically acceptable free-base nicotine, or nicotine salt, or nicotine salt hydrate. Useful nicotine salts or nicotine salt hydrates include nicotine pyruvate, nicotine citrate, nicotine aspartate, nicotine lactate, nicotine bitartrate, nicotine salicylate, nicotine fumarate, nicotine mono-pyruvate, nicotine glutamate or nicotine hydrochloride, for example. The compound combining with nicotine to form the salt or salt hydrate may be chosen based on its expected pharmacological effect.

The nicotine particles preferably include an amino acid. Preferably, the amino acid may be leucine such as L-leucine. Providing an amino acid such as L-leucine with the particles comprising nicotine, may reduce adhesion forces of the particles comprising nicotine and may reduce attraction between nicotine particles and thus reduce agglomeration of nicotine particles.

Similarly, adhesion forces to particles comprising flavour may also be reduced thus agglomeration of nicotine particles with flavour particles is also reduced. The powder system described herein thus may be a free-flowing material and possess a stable relative particle size of each powder component even when the nicotine particles and the flavour particles are combined.

Preferably, the nicotine may be a surface modified nicotine salt where the nicotine salt particle comprises a coated or composite particle. A preferred coating or composite material may be L-leucine. One particularly useful nicotine particle may be nicotine bi 5 tartrate with L-leucine.

The powder system may include a population of flavour particles. The flavour particles may have any useful size distribution for inhalation delivery selectively into the mouth or buccal cavity of a user.

The powder system may have at least about 40 percent, or at least about 60 percent, or at least about 80 percent, by weight of the population of flavour particles of the powder system comprised in particles having a particle size of about 20 micrometres or greater. The powder system may have at least about 40 percent or at least about 60 percent, or at least about 80 percent, by weight of the population of flavour particles of the powder system comprised in particles having a particle size of about 50 micrometres or greater. The powder system may have at least about 40 percent or at least about 60 percent, or at least about 80 percent, by weight of the population of flavour particles of the powder system comprised in particles having a particle size in a range from about 50 micrometres to about 150 micrometres.

The particles comprising flavour may include a compound to reduce adhesion forces or surface energy and resulting agglomeration. The flavour particle may be surface modified with an adhesion reducing compound to form a coated flavour particle. One preferred adhesion reducing compound may be magnesium stearate. Providing an adhesion reducing compound such as magnesium stearate with the flavour particle, especially coating the flavour particle, may reduce adhesion forces of the particles comprising flavour and may reduce attraction between flavour particles and thus reduce agglomeration of flavour particles. Thus, agglomeration of flavour particles with nicotine particles may also be reduced. The powder system described herein thus may possess a stable relative particle size of the particles comprising nicotine and the particles comprising flavour even when the nicotine particles and the flavour particles are combined. The powder system preferably may be free flowing.

Conventional formulations for dry powder inhalation contain carrier particles that serve to increase the fluidization of the active particles since the active particles may be too small to be influenced by simple airflow though the inhaler. The powder system may comprise carrier particles. These carrier particles may be a saccharide such as lactose or mannitol that may have a particle size greater than about 50 micrometres. The carrier particles may be utilized to improve dose uniformity by acting as a diluent or bulking agent in a formulation.

The powder system utilized with the nicotine powder delivery system described herein may be carrier-free or substantially free of a saccharide such as lactose or mannitol.

Being carrier-free or substantially free of a saccharide such as lactose or mannitol may allow the nicotine to be inhaled and delivered to the user's lungs at inhalation or airflow rates that are similar to typical smoking regime inhalation or airflow rates.

The nicotine particles and a flavour may be combined in a single capsule. As described above, the nicotine particles and a flavour may each have reduced adhesion forces that result in a stable particle formulation where the particle size of each component does not substantially change when combined. Alternatively, the powder system includes nicotine particles contained within a single capsule and the flavour particles contained within a second capsule.

The nicotine particles and flavour particles may be combined in any useful relative amount so that the flavour particles are detected by the user when consumed with the nicotine particles.

Preferably, the nicotine particles and flavour particles form at least about 90 weight-percent or at least about 95 weight-percent or at least about 99 weight-percent or 100 weight-percent of the total weight of the powder system.

A distal end portion of the semifinished inhaler article may comprise a deformable element. The deformable element may be a cardboard tube.

The term ‘deformable’ should be understood to mean that the shape of the deformable element is changeable. The deformation of the deformable element may include elastic deformation, where the deformable element reverts back to the closed configuration in the absence of a force being applied to it. Alternately, the deformation of the deformable element may include plastic deformation where the deformable element is held in the open configuration after the application of a force.

At least a portion of the deformable element may be formed of a foldable material. The deformable element may comprise a fan fold. At least a portion of the deformable element may be formed of cellulosic material. At least a portion of the deformable element may be formed of paper.

Advantageously, forming the deformable element of a foldable material allows the deformable element to be breached or opened reliably. A foldable material may also improve the assembly of the capsule cavity and provide for high-speed assembly of the inhaler article. Advantageously, the deformable element formed of cellulose material or paper is substantially biodegradable and may reduce the environmental impact of the inhaler article.

The deformable element may define at least a portion of a longitudinal sidewall of a cavity of the inhaler article. The cavity may hold a capsule. The deformable element may define a majority of the capsule cavity. The deformable element may define the upstream boundary and the sidewalls of the capsule cavity.

Advantageously, the deformable element may provide a protective cover or hygiene barrier for the retained capsule and inhaler article prior to consumption of the inhaler article. A wrapping layer may circumscribe the mouthpiece element and the deformable element. A wrapping layer may join the mouthpiece element, capsule cavity, and the deformable element in serial axial abutment. The deformable element may extend beyond the wrapping layer. The deformable element may extend beyond the wrapping layer in a range from about 0.5 millimetres to about 5 millimetres, or from about 1 millimetre to about 4 millimetres, or about 2 millimetres to about 3 millimetres. The wrapping layer may be formed of a cellulose material or paper.

Advantageously, a wrapping layer formed of cellulose material is substantially biodegradable and may reduce the environmental impact of the inhaler article. Joining inhaler article elements with a wrapping layer provides for high-speed assembly of the inhaler article.

The capsule cavity and deformable element may have substantially equal inner diameters in a range from about 6 millimetres to about 8 millimetres.

The capsule may contain pharmaceutically active particles. For instance, the pharmaceutically active particles may comprise nicotine. The pharmaceutically active particles may have a mass median aerodynamic diameter of about 5 micrometres or less, or in a range from about 0.5 micrometres to about 4 micrometres, or in a range from about 1 micrometres to about 3 micrometres.

The terms ‘proximal’ and ‘distal’ are used to describe the relative positions of components, or portions of components of the inhaler article or system. Inhaler articles, according to the invention have a proximal end. In use, the nicotine particles exit the proximal end of the inhaler article for delivery to a user. The inhaler article has a distal end opposing the proximal end. The proximal end of the inhaler article may also be referred to as the mouth end.

The inhaler article may resemble a smoking article or cigarette in size and shape. The inhaler article may have an elongated body extending along the longitudinal axis of the inhaler article. The inhaler body may have a substantially uniform outer diameter along the length of the elongated body. The inhaler article may have a circular cross-section that may be uniform along the length of the elongated body. The inhaler body may have an outer diameter in a range from about 6 millimetres to about 10 millimetres, or from about 7 millimetres to about 10 millimetres, or about 7 millimetres to about 9 millimetres, or about 7 millimetres to about 8 millimetres or about 7.3 millimetres. The inhaler article may have a length (along the longitudinal axis) in a range from about 40 millimetres to about 80 millimetres, or from about 40 millimetres to about 70 millimetres, or about 40 millimetres to about 50 millimetres, or about 48 millimetres.

The inhaler article may comprise a mouthpiece element. The mouthpiece element may be located downstream of the capsule cavity and may extend from the capsule cavity to the mouthpiece end of the inhaler article. The mouthpiece element may have a length in a range from about 10 millimetres to about 30 millimetres, preferably from about 15 millimetres to about 25 millimetres and more preferably from about 20 millimetres to about 22 millimetres. The mouthpiece element may have a diameter in a range from about 6 millimetres to about 10 millimetres, or from about 7 millimetres to about 10 millimetres, or about 7 millimetres to about 9 millimetres, or about 7 millimetres to about 8 millimetres or about 7.1 millimetres.

The mouthpiece element may have a filtering function. The mouthpiece element may comprise a filter element. The filter element may extend substantially over the full length of the mouthpiece element.

The deformable element may be configured to deform and expose the capsule cavity. The deformable element may be configured to be breached or opened to expose the capsule cavity. The deformable element may be configured to expose substantially the entire open diameter of the capsule cavity. The deformable element may be configured to expose the entire open diameter of the capsule cavity.

The deformable element may define at least a portion of a longitudinal sidewall of the capsule cavity. The deformable element may define a majority of the capsule cavity. The deformable element may define a closed distal end or upstream end of the capsule cavity.

The deformable element may be formed of cellulosic material. At least a portion of the deformable element may be formed of paper. The deformable element may provide a barrier to reduce or prevent contaminants or foreign material from entering the capsule cavity.

The capsule cavity sidewall may extend parallel with the longitudinal axis of the inhaler article. The deformable element may define a closed distal end or upstream end of the capsule cavity and at least a portion of the capsule cavity sidewall.

The deformable element may define a tubular element having a closed upstream end. The deformable element may define a closed distal end or upstream end of the capsule cavity and at least 50 percent of the capsule cavity sidewall. The deformable element may define a closed distal end or upstream end of the capsule cavity and at least 75 percent of the capsule cavity sidewall. The deformable element may define a closed distal end or upstream end of the capsule cavity and the entire capsule cavity sidewall. The deformable element may define the entire capsule cavity except for the downstream boundary surface defined by the mouthpiece element. The deformable element may be a paper layer extending from the mouthpiece element to the closed upstream end.

Inhalation air may flow through the center of the deformable element directly into the capsule cavity once the deformable element is breached or opened. The deformable element may have a diameter that is substantially equal to the inner diameter of the capsule cavity.

The deformable element may have an outer diameter in a range from about 6 millimetres to about 8 millimetres or from about 7.0 millimetres to about 7.1 millimetres. The deformable element may have an inner diameter in a range from about 6 millimetres to about 7.2 millimetres or from about 6.5 millimetres to about 6.7 millimetres.

The deformable element may be formed of paper. The deformable element may be formed of one or more paper layers. The deformable element may be formed of paper having a weight in a range of about 50 grams per square meter to about 150 grams per square meter, or from about 75 grams per square meter to about 125 grams per square meter, or from about 90 grams per square meter to about 110 grams per square meter.

The deformable element may have a thickness in a range from about 50 micrometres about 200 micrometres, or from about 100 micrometres to about 150 micrometres, or from about 110 micrometres to about 130 micrometres.

Once breached or opened, the deformable element may define an opening having an open diameter that is at least about 80 percent or at least about 90 percent of the diameter of the capsule cavity.

The deformable element may be easily breached to allow inhalation air to enter the capsule cavity. For instance, the deformable element may be configured to breach upon manual insertion of the inhaler article into a user-holder device by a user without the use of additional tools for assisting the application of force by a user. The deformable element may breach or open to expose substantially the entire upstream end of the capsule cavity. The deformable element may provide a protective cover or hygiene barrier for the retained capsule and inhaler article prior to consumption of the inhaler article.

A wrapping layer may define the body of the inhaler article. The wrapping layer may circumscribe the mouthpiece element and the deformable element. The wrapping layer may join the mouthpiece element and the deformable element. The wrapping layer may join the mouthpiece element, and deformable element in serial axial abutment. The wrapping layer may be formed of a cellulose material.

The deformable element may extend beyond the wrapping layer. The deformable element may extend beyond the wrapping layer in a range from about 0.5 millimetres to about 5 millimetres, or from about 1 millimetre to about 4 millimetres, or about 2 millimetres to about 3 millimetres.

The method may comprise, before the step of at least partially closing the distal end of the aligned semifinished inhaler article, a step of pre-treating a distal end portion of the semifinished inhaler article to obtain a pre-treated portion with reduced structural stability.

The pre-treatment may comprise providing a pre-treating station. The pre-treating station may include a processing head for creasing, cutting or scoring the distal end of the deformable tubular element.

The pre-treating step may comprise crimping the edge of the distal end of the deformable tubular element. Upon crimping the edge of the deformable tubular element is folded along one or more lines running essentially parallel to the axial direction of the inhaler article.

The pre-treating step may comprise cutting the edge of the distal end of the deformable tubular element along one or more lines running generally parallel to the axial direction of the inhaler article.

The pre-treating step may comprise scoring the edge of the distal end of the deformable tubular element along one or more lines running generally parallel to the axial direction of the inhaler article. Upon scoring the deformable element may be provided with a discontinuous cutting line.

The length of the crimping, scoring or cutting lines may be in a range from 0.5 to 5 millimeter, preferably from about 1 to 4 millimeters, and preferably from about 2.5 to 3.5 millimeters. Generally, the length of these lines determines the length of the pre-treated portion with reduced structural stability.

The required length of the pre-treated portion depends on the diameter of the inhaler article.

Typical inhaler articles may have a diameter of 7.2 millimeters. For such articles the useful length of the pretreated portion may be at least about 3 millimeters and may be at most equal to the radius (3.6 millimeters). With a pre-treated portion of such dimensions, a sufficient closure of the distal end of the deformable tubular element may be achieved.

During the pre-treating step, the distal end of the deformable tubular element may be provided with 4 to 15 creasing, cutting or scoring lines. Preferably, the deformable tubular element may be provided with 6 to 12 creasing, cutting or scoring lines. Preferably, the deformable tubular element may be provided with 8 to 10 creasing, cutting or scoring lines.

The more creasing, cutting or scoring lines are provided, the better the deformable tubular element may be folded into a cylindrical form. However, with increasing number of creasing, cutting or scoring lines, the complexity of the folding process increases. For typical paper material used in manufacturing the inhaler article having a diameter of about 7.2 millimeters a number of 8 to 10 creasing, cutting or scoring lines have proven to yield best results.

In general, the creasing, cutting or scoring lines may be formed such as to extend parallel to the longitudinal axis of the deformable tubular element. However, these lines can also be formed to extend under any desired angle with respect to the longitudinal axis of the inhaler article. These lines may be formed to extend under an angle of between 0 to 45 degrees with respect to the longitudinal axis of the inhaler article.

The step of at least partially closing the distal end of the semifinished inhaler article may comprise folding a distal end portion of the semifinished inhaler article inwards by at least 90 degrees. The distal end portion of the semifinished inhaler article may be folded inwardly by an angle of between 90 and 110 degrees.

In some embodiments, the distal ends of the semifinished inhaler articles are to be completely closed by folding a distal end portion of the semifinished inhaler articles inwards by at least 90 degrees. Without the step of aligning the semifinished inhaler articles as disclosed herein, some of the shorter, or inadequately positioned, semifinished inhaler articles may disadvantageously not be completely closed due to a shorter distal end portion being folded.

In some embodiments, the distal ends of the semifinished inhaler articles are to be partially closed by folding a distal end portion of the semifinished inhaler articles inwards by at least 90 degrees such that a central aperture at the center of the folded distal end remains open. Without the step of aligning the semifinished inhaler articles as disclosed herein, some of the shorter semifinished inhaler articles may disadvantageously have a wider aperture due to a shorter distal end portion being folded. Without the step of aligning the semifinished inhaler articles as disclosed herein, some of the longer semifinished inhaler articles may disadvantageously have a smaller aperture due to a longer distal end portion being folded. This may result in a non-uniform appearance of the folded distal ends.

The distal ends of all semifinished inhaler articles received in the rotating drum may be closed by applying the same force.

The one or more rotating drums may be arranged such that the longitudinal axes of the inhaler articles lie within a horizontal plane when being received in the slots of the rotating drum.

According to an embodiment, the method for manufacturing inhaler articles, comprises steps of providing a semifinished inhaler article comprising a longitudinal axis, a proximal end, and an open distal end, providing a rotating drum comprising a plurality of slot elements with movable end faces, inserting the semifinished inhaler article into a slot element, rotating the rotating drum such that the semifinished inhaler article is located in proximity to an alignment element provided adjacent to a first segment of the rotating drum, moving the movable end face of the slot element against the proximal end of the semifinished inhaler article to push the semifinished inhaler article along its longitudinal axis until the open distal end of the semifinished inhaler article contacts the alignment element, fixing the movable end face in position, rotating the rotating drum such that the semifinished inhaler article is located in proximity to a closing station provided adjacent to a second segment of the rotating drum, and at least partially closing the distal end of the semifinished inhaler article. The method may comprise, after the step fixing the movable end face in position, steps of, rotating the rotating drum such that the semifinished inhaler article is located in proximity to a filling station provided adjacent to a third segment of the rotating drum, and inserting a capsule into the open distal end of the semifinished inhaler article. The third section of the rotating drum may be located between the first and second sections.

As used herein, the term ‘method for manufacturing inhaler articles’, may refer to a method for manufacturing a completely finished inhaler article, or may refer to a method for manufacturing a sub-unit of an inhaler article, or a double-length inhaler article.

As used herein, the term ‘inhaler article’, may refer to any type of inhaler article known to the skilled person. The term ‘inhaler article’, may refer to an aerosol-generating article comprising an aerosol-generating substrate which is to be heated to produce and deliver inhalable aerosol to a user. The term ‘inhaler article’, may refer to a dry powder inhaler.

The finished inhaler article may comprise a body, a capsule cavity holding a capsule, a mouthpiece element at a proximal end, and a deformable tubular element having an at least partially closed distal end.

The invention further relates to a package comprising a plurality of inhaler articles manufactured by the method described herein.

The package may comprise between 5 and 40 inhaler articles, preferably between 10 and 30 inhaler articles, more preferably between 15 and 25 inhaler articles, and most preferably between 18 and 22 inhaler articles.

The at least partially closed ends of the inhaler articles in the package may have a uniform appearance.

A difference in a diameter of a central aperture of a partially closed distal end of the inhaler articles in the package may be less than 20%, preferably less than 15%, more preferably less than 10%, more preferably less than 5%, more preferably less than 2%, and most preferably less than 1%.

The invention further relates to a rotating drum for an apparatus for manufacturing inhaler articles. The rotating drum comprises a plurality of circumferentially arranged slot elements. Each slot element comprises a recess for insertion of a semifinished inhaler article and a movable end face for adjusting a longitudinal position of the semifinished inhaler article within the recess. The rotating drum comprises a releasable fixing means for holding the movable end faces in position. The releasable fixing means may comprise individual releasable fixing elements for each movable end face. Plural movable end faces may be fixed by a common releasable fixing element of the releasable fixing means.

The invention further relates to an apparatus for manufacturing inhaler articles, comprising a first rotating drum as described herein and an alignment element provided in proximity to a portion of the rotating drum.

The apparatus may comprise a second rotating drum as described herein arranged in reverse-orientation with respect to the first rotating drum and adjacent to the first rotating drum.

As used herein, the term ‘nicotine’ refers to nicotine and nicotine derivatives such as free-base nicotine, nicotine salts and the like.

As used herein, the term ‘flavourant’ or ‘flavour’ refers to organoleptic compounds, compositions, or materials that alter and are intended to alter the taste or aroma characteristics of nicotine during consumption or inhalation thereof.

Below, there is provided a non-exhaustive list of non-limiting examples. Any one or more of the features of these examples may be combined with any one or more features of another example, embodiment, or aspect described herein.

Example A: A method for manufacturing inhaler articles, comprising steps of
providing a semifinished inhaler article comprising a longitudinal axis, a proximal end, and an open distal end;
   providing a rotating drum comprising a plurality of slot elements with movable end faces;
inserting the semifinished inhaler article into a slot element;
providing an alignment element in proximity to the semifinished inhaler article;
moving the movable end face of the slot element against the proximal end of the semifinished inhaler article to push the semifinished inhaler article along its longitudinal axis until the open distal end of the semifinished inhaler article contacts the alignment element;
fixing the movable end face in position;
providing a closing station in proximity to the semifinished inhaler article; and
   at least partially closing the distal end of the semifinished inhaler article.

Example B: The method according to Example A, wherein the movable end face of the slot element is moved by means of a spring element coupled to the movable end face.

Example C: The method according to Example A or Example B, comprising, after the step of at least partially closing the distal end of the semifinished inhaler article, steps of
removing the semifinished inhaler article from the slot element; and
retracting the movable end face of the slot element.

Example D: The method according to Example C, wherein the step of removing the semifinished inhaler article from the slot element comprises transferring the inhaler article to a second rotating drum which is reverse-orientated relative to the first rotating drum but which is otherwise identical to the first rotating drum.

Example E: The method according to Example D, wherein the proximal end of the semifinished inhaler article is an open proximal end, and wherein the method further comprises at least partially closing the proximal end of the semifinished inhaler article at the second rotating drum.

Example F: The method according to any of the preceding examples, comprising, after the step fixing the movable end face in position, steps of,
providing a filling station in proximity to the semifinished inhaler article; and
   inserting a capsule into the open distal end of the semifinished inhaler article.

Example G: The method according to Example F, wherein the capsule comprises nicotine.

Example H: The method according to Example For Example G, wherein the capsule comprises a dry powder.

Example I: The method according to any of the preceding examples, wherein a distal end portion of the semifinished inhaler article comprises a deformable cardboard tube.

Example J: The method according to any of the preceding examples, comprising, before the step of at least partially closing the distal end of the semifinished inhaler article, a step of pre-treating a distal end portion of the semifinished inhaler article to obtain a pre-treated portion with reduced structural stability.

Example K: The method according to any of the preceding examples, wherein the step of at least partially closing the distal end of the semifinished inhaler article comprises folding a distal end portion of the semifinished inhaler article inwards by at least 90 degrees.

Example L: The method according to any of the preceding examples, wherein the distal ends of all semifinished inhaler articles received in the rotating drum are closed by applying the same force.

Example M: A package comprising a plurality of inhaler articles manufactured by the method of any of Examples A to L.

Example N: The package according to Example M, comprising between 5 and 40 inhaler articles, preferably between 10 and 30 inhaler articles, more preferably between 15 and 25 inhaler articles, and most preferably between 18 and 22 inhaler articles.

Example O: The package according to Example M or Example N, wherein the at least partially closed ends of the inhaler articles have a uniform appearance.

Example P: The package according to any of Examples M, N, and O, wherein a difference in a diameter of a central aperture of a partially closed distal end of the inhaler articles is less than 20%, preferably less than 15%, more preferably less than 10%, more preferably less than 5%, more preferably less than 2%, and most preferably less than 1%.

Example Q: A rotating drum for an apparatus for manufacturing inhaler articles, comprising
a plurality of circumferentially arranged slot elements, each slot element comprising a recess for insertion of a semifinished inhaler article and a movable end face for adjusting a longitudinal position of the inhaler article within the recess; and
a releasable fixing means for holding the movable end faces in position.

Example R: An apparatus for manufacturing inhaler articles, comprising
a first rotating drum according to Example Q, and
an alignment element provided in proximity to a portion of the rotating drum.

Example S: The apparatus of Example R, comprising a second rotating drum according to Example Q arranged in reverse-orientation with respect to the first rotating drum and adjacent to the first rotating drum.

Features described in relation to one embodiment may equally be applied to other embodiments of the invention.

The invention will be further described, by way of example only, with reference to the accompanying drawings in which:

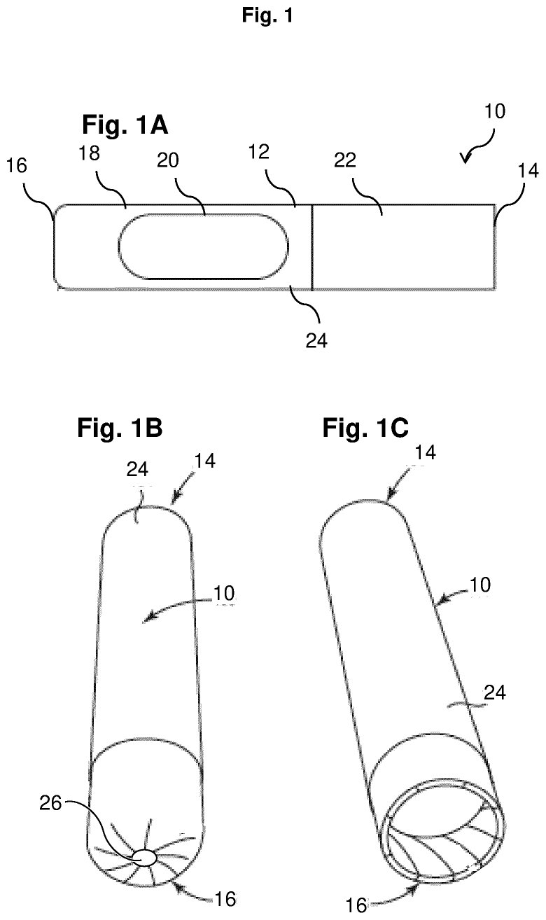

FIGS. 1A-1C show an illustrative inhaler article;

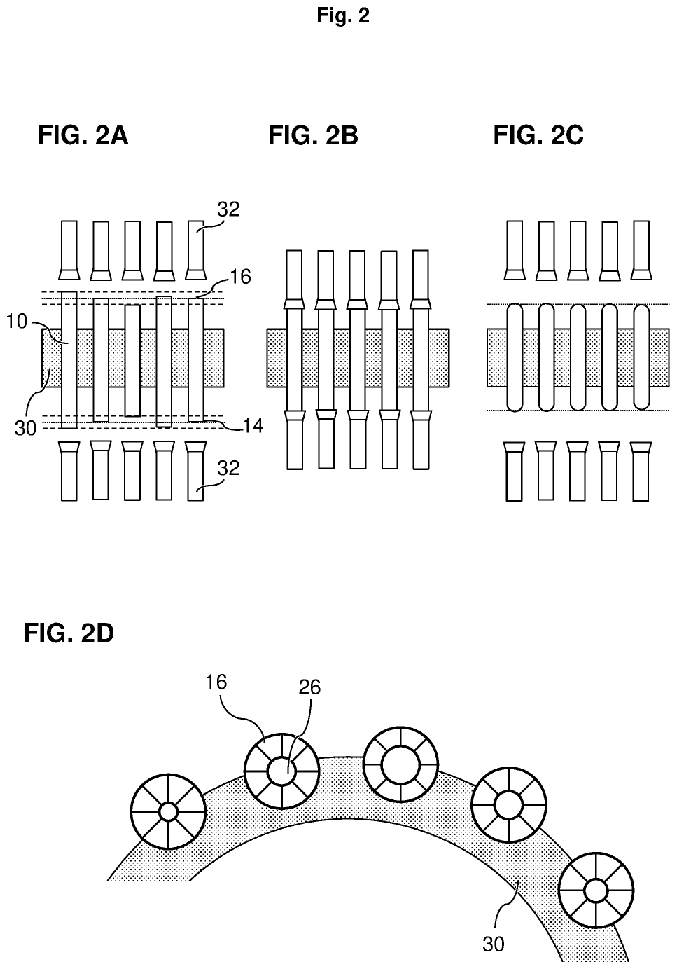

FIGS. 2A-2D show steps of closing open ends of double-length inhaler articles without alignment;

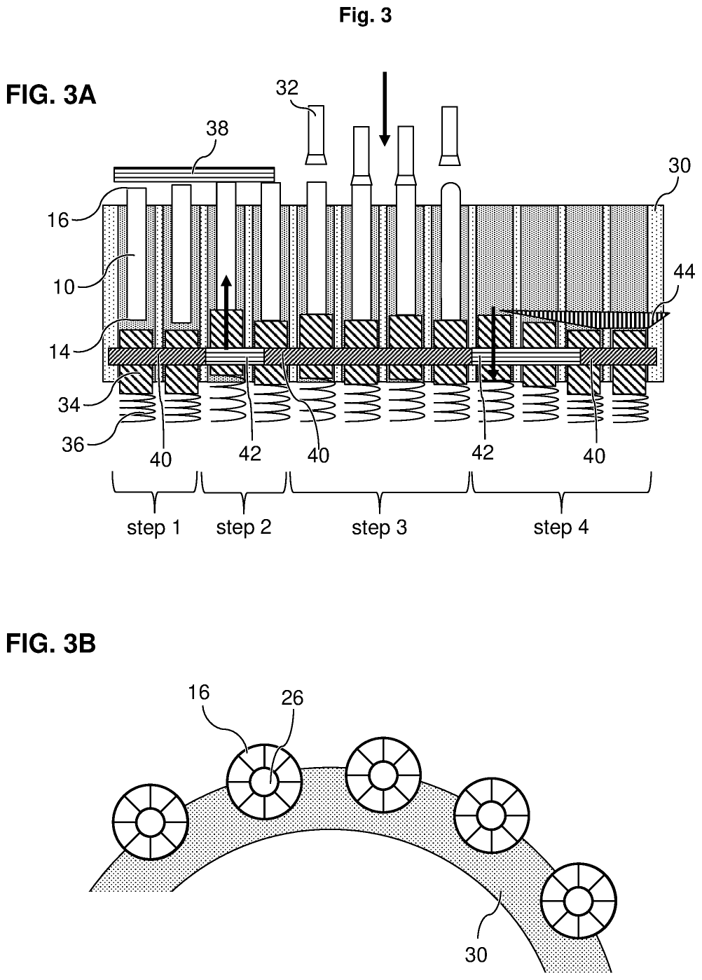

FIGS. 3A and 3B show steps of alignment and closing of inhaler articles; and

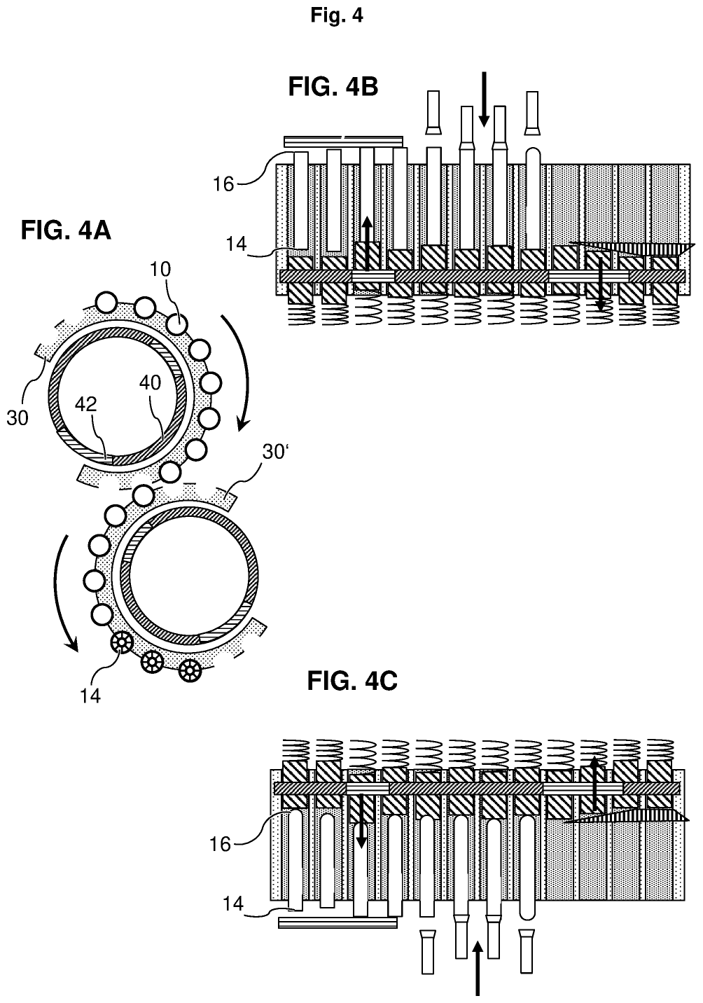

FIGS. 4A-4C show steps of alignment and closing of double-length inhaler articles.

FIG. 1A is a cross-sectional schematic diagram of an illustrative inhaler article 10. The inhaler article 10 includes a body 12 extending along a longitudinal axis of the inhaler article 10 from a proximal end 14 to a distal end 16, a capsule cavity 18 and a capsule 20 retained within the capsule cavity 18. The body 12 comprises a paper material wrapped around a mouthpiece element 22 forming a deformable tubular element 24. The deformable tubular element 24 defines the capsule cavity 18, which is bounded downstream by mouthpiece element 22 and which is bounded upstream by the at least partially closed distal end 16 of the deformable tubular element 24.

In the embodiment of FIG. 1 the deformable tubular element 24 is formed of paper having a thickness of about 125 micrometers and a basis weight of about 100 grams per square meter. The illustrated inhaler article 10 has a mouthpiece element length of about 20 mm and the deformable tubular element 24 has a length of about 45 mm with an outer uniform diameter of about 7.2 mm.

FIG. 1B is a front perspective view of the illustrative inhaler article 10 wherein the distal end 16 of the deformable tubular element 24 is partially closed with the exception of a central aperture 26. The deformable element 24 is folded back onto itself forming overlapping pie shaped sections partially closing the distal end 16 of the capsule cavity 18.

FIG. 1C is a front perspective view of the illustrative inhaler article with a deformable tubular element 24 wherein the distal end 16 is opened. The folded sections of the distal end 16 of the deformable tubular element 24 may be opened to expose the capsule cavity 18. For opening the distal end 16, the deformable tubular element 24 may be inserted into an appropriate user-holder device, not described herein. After the folded sections of the distal end 16 of the deformable element 24 are opened, an aperture for receiving swirling or rotating inhalation airflow is formed.

FIGS. 2A-2D show steps of closing open ends of double-length inhaler articles 10 without alignment of the present invention. FIGS. 2A-2C show the articles 10 arranged within slots of a rotating drum 30 in top view.

FIG. 2A shows semifinished double-length inhaler articles 10 with open distal ends 16 and open proximal ends 14. Due to manufacturing tolerances, the semifinished inhaler articles 10 differ in lengths. As a consequence, the relative longitudinal positions of the open distal ends 16 and the open proximal ends 14 of the articles 10 in the rotating drum 30 differ. This is indicated by two pairs of each three dotted lines in FIG. 2A. The dotted centered lines (small dots) each indicate the desired longitudinal position of the open distal ends 16 and the open proximal ends 14, respectively according to the nominal length of the articles 10. Each the upper and lower dotted lines (large dots) indicate the differences in positions for longer and shorter articles 10, respectively. Folding heads 32 which form part of a closing station are also shown in FIG. 2A.

FIG. 2B indicates a subsequent step, where the folding heads 32 have been moved towards the semifinished double-length inhaler articles 10 in order to at least partially close the open ends 14, 16 of the articles 10 by folding each a distal end portion and a proximal end portion of the semifinished inhaler articles 10 inwards by at least 90 degrees.

FIG. 2C indicates a subsequent step, where the folding heads 32 have been retracted after having closed the ends 14, 16 of the articles 10.

FIG. 2D shows the partially closed distal ends 16 in side view. A portion of the rotating drum 30 is also shown. The central apertures 26 of the partially closed distal ends 16 differ in their sizes. In other words, the inner diameters of the central apertures 26 differ. This is caused by the different heights of the distal ends 16 before folding (FIG. 2A) which, in turn, result in different lengths of the folded distal end portions. The result thus is a non-uniform appearance of the partially closed distal ends 16 of the inhaler articles 10 as shown in FIG. 2D. The partially closed proximal ends 14 on the other side (not shown) have a similar non-uniform appearance.

FIG. 3A shows steps of alignment and closing of semifinished inhaler articles in accordance to an embodiment of the invention. FIG. 3A shows the articles 10 arranged within slots of a rotating drum 30 in top view. The rotating drum 30 is arranged such that the longitudinal axes of the inhaler articles lie within a horizontal plane.

Step 1 shows the semifinished inhaler articles 10 inserted into slot elements of the rotating drum 30. Each slot element comprises a movable end face 34 mounted to a spring element 36. An alignment element 38 is provided in proximity to the semifinished inhaler articles 10. A fixing means is in its fixing state 40 to hold the movable end faces 34 in a retracted position.

In step 2, the fixing means is brought into its loosened state 42 such that the spring elements 36 move the movable end faces 34 of the slot elements against the proximal end 14 of the semifinished inhaler articles 10 to push the semifinished inhaler articles 10 along their longitudinal axes until the open distal ends 16 of the semifinished inhaler articles 10 contact the alignment element 38. The movement is indicated by an arrow. Thereby, the open distal ends 16 of the semifinished inhaler articles 10 are aligned to the exact same height. The fixing means is then brought into its fixing state 40 to hold the movable end faces 34 in the advanced positions.

In step 3, a folding station with folding heads 32 is provided in proximity to the semifinished inhaler articles 10. The folding heads 32 are moved towards the distal ends 16 of the semifinished inhaler articles 10 in order to at least partially close the open distal ends 16 of the articles 10 by folding a distal end portion of the articles 10 inwards by at least 90 degrees. The movement of the folding heads 32 is indicated by an arrow. Then, the folding heads 32 are retracted again after having partially closed the distal ends 16 of the articles 10.

The articles 10 with partially closed distal ends 16 are then removed from the slots of the rotating drum 30. This removal step is not shown in FIG. 3A.

In step 4, the articles 10 have been removed from the slots and the advanced movable end faces 34 are brought back from the advanced positions into the retracted position. The fixing means is brought into the loosened state 42 and a retracting element 44 retracts the movable end faces 34. The retracting movement is indicated by an arrow. The fixing means is then brought into the fixing state 40 again.

FIG. 3B shows the partially closed distal ends 16 in side view. A portion of the rotating drum 30 is also shown. The central apertures 26 of the partially closed distal ends 16 do not differ in their sizes. In other words, the inner diameters of the central apertures 26 do not differ. This is achieved by the same heights of the distal ends 16 before folding (step 2 in FIG. 3A) which, in turn, result in same lengths of the folded distal end portions. The result thus is a uniform appearance of the distal ends 16 of the inhaler articles 10 as shown in FIG. 3B.

FIGS. 4A-4C show steps of alignment and closing of double-length inhaler articles 10 in accordance to an embodiment of the invention.

FIG. 4A shows the articles 10 being transferred between a first rotating drum 30 and a second rotating drum 30′ in side view. The second rotating drum 30′ is arranged in reverse-orientation with respect to the first rotating drum 30 and adjacent to the first rotating drum 30. Arrows indicate the rotating direction of the rotating drums 30, 30′. It also indicated in FIG. 4A at which positions the fixing means of the rotating drums 30, 30′ is in its fixing state 40, or in its loosened state 42.

FIG. 4B shows aligning of the semifinished double-length inhaler articles 10 with open distal ends 16 and open proximal ends 14 and at least partially closing the aligned distal ends 16 at the first rotating drum 30 in top view. The process is conducted in four steps as described above with reference to FIG. 3A.

FIG. 4C shows aligning of the semifinished double-length inhaler articles 10 with closed distal ends 16 and open proximal ends 14 and at least partially closing the aligned proximal ends 14 at the second rotating drum 30′ in top view. The process is again conducted in four steps as described above with reference to FIG. 3A. The closed proximal ends 14 are indicated in FIG. 4A.

After folding both its ends, the double-length inhaler article 10 may be cut in the middle, to obtain two inhaler articles with closed distal ends. Cutting can be performed with conventional cutting devices.

1. A method for manufacturing inhaler articles, comprising steps of
providing a semifinished inhaler article comprising a longitudinal axis, a proximal end, and an open distal end;
providing a rotating drum comprising a plurality of slot elements with movable end faces;
inserting the semifinished inhaler article into a slot element;
providing an alignment element in proximity to the semifinished inhaler article;
moving the movable end face of the slot element against the proximal end of the semifinished inhaler article to push the semifinished inhaler article along its longitudinal axis until the open distal end of the semifinished inhaler article contacts the alignment element;
fixing the movable end face in position;
providing a closing station in proximity to the semifinished inhaler article; and
at least partially closing the distal end of the semifinished inhaler article.

2. The method according to claim 1, wherein the movable end face of the slot element is moved by means of a spring element coupled to the movable end face.

3. The method according to claim 1, comprising, after the step of at least partially closing the distal end of the semifinished inhaler article, steps of
removing the semifinished inhaler article from the slot element; and
retracting the movable end face of the slot element.

4. The method according to claim 3, wherein the step of removing the semifinished inhaler article from the slot element comprises transferring the inhaler article to a second rotating drum which is reverse-orientated relative to the first rotating drum but which is otherwise identical to the first rotating drum.

5. The method according to claim 4, wherein the proximal end of the semifinished inhaler article is an open proximal end, and wherein the method further comprises at least partially closing the proximal end of the semifinished inhaler article at the second rotating drum.

6. The method according to claim 1, comprising, after the step fixing the movable end face in position, steps of,
providing a filling station in proximity to the semifinished inhaler article; and
inserting a capsule into the open distal end of the semifinished inhaler article.

7. The method according to claim 6, wherein the capsule comprises nicotine.

8. The method according to claim 6, wherein the capsule comprises a dry powder.

9. The method according to claim 1, comprising, before the step of at least partially closing the distal end of the semifinished inhaler article, a step of pre-treating a distal end portion of the semifinished inhaler article to obtain a pre-treated portion with reduced structural stability.

10. The method according to claim 1, wherein the step of at least partially closing the distal end of the semifinished inhaler article comprises folding a distal end portion of the semifinished inhaler article inwards by at least 90 degrees.

11. A package comprising a plurality of inhaler articles manufactured by the method of claim 1.

12. The package according to claim 11, wherein the at least partially closed ends of the inhaler articles have a uniform appearance.

13. The package according to claim 11, wherein a difference in a diameter of a central aperture of a partially closed distal end of the inhaler articles is less than 20%, preferably less than 15%, more preferably less than 10%, more preferably less than 5%, more preferably less than 2%, and most preferably less than 1%.

14. The package according to claim 13, wherein a difference in a diameter of a central aperture of a partially closed distal end of the inhaler articles is less than 2%, preferably less than 1%.

15. A rotating drum for an apparatus for manufacturing inhaler articles, comprising a plurality of circumferentially arranged slot elements, each slot element comprising a recess for insertion of a semifinished inhaler article and a movable end face for adjusting a longitudinal position of the inhaler article within the recess; and
a releasable fixing means for holding the movable end faces in position.

16. An apparatus for manufacturing inhaler articles, comprising
a first rotating drum according to claim 15, and
an alignment element provided in proximity to a portion of the rotating drum.
